(19) United States
(12) Patent Application Publication
(10) Pub. No.: US 2008/0063709 A1
(43) Pub. Date: Mar. 13, 2008

(54) PHARMACEUTICAL COMPOSITIONS HAVING NOVEL SCORING PATTERNS

(76) Inventors: Elliot F. Hahn, North Miami, FL (US); Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/859,249

(22) Filed: Sep. 21, 2007

Related U.S. Application Data
(63) Continuation-in-part of application No. 11/735,268, filed on Apr. 13, 2007.
(60) Provisional application No. 60/826,665, filed on Sep. 22, 2006.
(60) Provisional application No. 60/791,834, filed on Apr. 13, 2006.
(60) Provisional application No. 60/792,601, filed on Apr. 17, 2006.
(60) Provisional application No. 60/792,933, filed on Apr. 18, 2006.
(60) Provisional application No. 60/861,898, filed on Nov. 30, 2006.

(51) Int. Cl. A61K 9/44; A61K 31/335; A61P 25/00

(52) U.S. Cl. 424/467; 514/450

(57) ABSTRACT

The invention provides novel scoring patterns for a wide variety of tablets that contain specific drugs or contain drugs in certain drug classes, preferably doxepin.

### CROSS-REFERENCE TO RELATED APPLICATIONS

This is a continuation of U.S. Provisional Application, Ser. No. 60/826,665, filed Sep. 22, 2006; and is a continuation-in-part of U.S. patent application, Ser. No. 11/735,268, filed Apr. 13, 2007, which is a continuation-in-part and claims the benefit of U.S. Provisional Application Ser. No. 60/791,834 filed Apr. 13, 2006; U.S. Provisional Application Ser. No. 60/792,601 filed Apr. 17, 2006; U.S. Provisional Application Ser. No. 60/792,933 filed Apr. 18, 2006; and U.S. Provisional Application Ser. No. 60/861,898 filed Nov. 30, 2006.

### BACKGROUND OF THE INVENTION

It is known to create a pharmaceutical tablet with one or more indentation, mark, or score (hereinafter, a score). Typically the score is a bisecting score, which provides an indication in the tablet of its divisibility into two substantially equal portions. Other patterns such as trisection, indicating divisibility into three portions, and quadrisection, indicating divisibility into four sections, are known but are far rarer than bisection. Reasons for this fact may include the observation that when a tablet is broken twice, any imprecision in the predicted dose in a portion resulting from the first breaking carries over and is additive to any such imprecision in the next breaking. It is well documented that breaking of scored tablets provides potentially significant variation in the precision of any dose provided in a resultant tablet portion, and in general provides doses of less accuracy than are produced by creating a smaller dose separately, as in a separately manufactured dosage form containing that lower dose.

As used herein, the term “quadrisected” (or, “quadrisect,” “quadrisection,” etc.) refers to a crossing scoring pattern or three parallel score marks, so that breaking the tablet precisely as guided by said two or three scores would in theory provide four equal tablettes, each containing one-fourth the mass/drug quantity as the whole tablet. This is differentiated from the scoring pattern utilized in Dividose® or Desyrel® (trazodone) and Buspar® (buspirone) in which two parallel scores on one surface of the tablet trisect the tablet, and a bisecting score is present on a different surface, typically an opposing surface.

Searches of tablets on the market in the U.S. and searches from international sources have produced the following relatively limited list of medications that have been scored other than in a bisected manner:
Known quadrisected tablets:
   Alprazolam (anti-anxiety)
   Captopril, enalapril, lisinopril (ACE inhibitors)
   Atropine, scopolamine (anti-cholinergics)
   Acetazolamide (carbonic anhydrase inhibitor)
   Chlorpropamide, glicazide (sulfonylurea hypoglycemics)
   Methadone (narcotic)
   Methylprednisolone (corticosteroid)
   Simethicone (anti-flatulent)
   Paroxetine and fluoxetine (antidepressants)
   Carbamazepine (anti-neuropathic agent, anti-convulsant)
   Tizanidine (antispasmodic)
   Paracetacod (narcotic)—combination product containing codeine and ascorbic acid
Known bisected/trisected tablets:
   Buspirone
   Trazodone

In view of the large number of tablet or tabletable products that have a 1:2:3 or a 1:2:4 milligram ratio for marketed strengths of manufactured dosage forms, certain benefits can be achieved by providing tablets having a unique scoring pattern that enables portions or sections of tablets broken along those unique scores to predictably and accurately be split into dosages of one-quarter, one-third, half, two-thirds, or three-quarters of the whole tablet. The benefits derived from compositions according to the subject invention include medical benefits.

On a product-by-product basis, good reasons for not providing more complex scoring patterns have apparently existed. For example, warfarin sodium is an anticoagulant provided as a bisected tablet. A study published in the Journal of the American Medical Association (JAMA) of “elderly” patients using tablet splitters found major variations were present in the sub-doses or tablet portions resulting from “halving” this scored tablet. Given the importance of accurate dosing with warfarin, it has, in the past, been important that quadrisection not be performed with manufactured warfarin product, lest it induce patients, aides, and other users to break the tablet into four pieces, as further “halving” of the two inaccurately divided “halves,” (i.e., “quartering” of the whole tablet) can magnify the error caused by the initial “halving” of the tablet. Thus it has not been taught or suggested to produce a quadrisected warfarin tablet.

In another example, it is known that anticonvulsants frequently undergo dose adjustment, and that accurate dosing is important. The well known anticonvulsant Lamictal® (lamotrigine) is provided as scored tablets in the U.S. but per the Product Information (PI, or “label”) for Lamictal, the tablets are to be taken whole and not broken. It has therefore not been taught or suggested to provide a quadrisect pattern on a Lamictal tablet.

In many other cases, such as antipsychotics and various drugs for psychiatric, neurologic, and cardiovascular uses, trisection or quadrisection has within a class either never been done, or has been done rarely without a teaching or suggestion to break those tablets into portions according to the scoring pattern provided on those tablets.

Along with these medical benefits, which include a manufacturer's avoidance of inappropriate dosing by inducing or implicitly or explicitly recommending breaking a tablet and then breaking one or more of the tablettes formed by said first breaking, another reason that certain tablets have not been provided with more than a bisecting score may involve the physical characteristics or properties of the tablets. For example, hardness and other breaking characteristics of a particular drug formulation available in the prior art may contribute to the unsuitability of such complex scoring, or perhaps even for bisecting.

Another reason that certain tablets are not scored, and are especially inappropriate for complex scoring beyond bisecting, involves the need to film-coat the tablet. Thus, certain preferred compositions of the subject invention can provide a benefit for permitting unique scoring patterns on or in tablets that may be coated.

It is noted that apparently the most recent introduction of a trisected or quadrisected tablet was that of paroxetine, for which only the 40 mg tablet is reported to be quadrisected, per its label as marketed in South Africa (“Parax” brand). Thus it appears that recent practice has been against trisection or quadrisection of pharmaceutical tablets. Because there are significant potential adverse consequences from inducing a patient to break a pharmaceutical tablet into three or four pieces, because of such reasons as are described above, most prominently among them the inaccurate dosing that can result from such breaking, those of reasonable skill in the pharmaceutical arts have ceased manufacture of such products.

### SUMMARY OF THE INVENTION

The invention provides dosage forms, e.g., tablets, comprising novel scoring patterns for a wide variety products containing specific active pharmaceutical ingredients (APIs). An object of the invention is to allow enhanced dosage flexibility over the current art, in a manner that benefits the practice of medicine and pharmaceutics. Making use of advances in dosing flexibility, the invention involves all pharmaceutical products such as tablets or capsules that have not, heretofore, been made in a readily divisible dosage form having a particular score pattern. The invention is especially concerned with pharmaceutical tablets for which more than one useful dose is known, for example, where three useful doses representing a ⅓, ⅔, and whole dose are known for trisected tablets, or where a ¼, ½, and whole dose are known for quadrisected tablets, or the like.

The Physicians' Desk Reference, Section 3, 2006 Edition, (PDR 2006) lists numerous classes of drugs and drug products containing one or more specific APIs. The relevant portions of the PDR 2006 are incorporated herein by reference. Except for the specific examples of trisected and quadrisected tablets listed above, the drug classes that involve solid oral dosage forms, and the specific drug products that involve solid oral dosage forms, are claimed for the subject invention. There is thus no limitation to the classes or specific names of drugs that fall within the scope of the invention. The drugs or APIs provided in the examples below are exemplary only.

Among the most preferred embodiments of the invention are tablets that provide predictably accurate doses when broken into tablet portions. For example, tablets comprising unitary segments and an inactive substrate or base layer, as described by Solomon and Kaplan in WO 2005/112900 and WO 2006/038916, or layered or segmented tablets as described in WO 2005/112870, WO 2005/112897, and WO 2005/112898. Accurately breakable tablets are also described in US Published Application No. 2002/0052411, U.S. Pat. Nos. 6,488,939, 6,692,765, 6,342,248, 5,756,124, 5,520,929, 4,258,027, 3,723,614, 3,336,200, Re. 29077, and UK Patent GB 2351234A, though certain of the tablet designs described therein may not be suitable for other than bisected tablets.

Among the tablets more preferred for the invention are, without limitation, drug products of the following classes:

Anticoagulants, antihypertensives, antipsychotics, tranquilizers, anticonvulsants, hypoglycemics, antidepressants, psychotherapeutic agents such as dibenzoxepin tricyclic compounds, e.g., doxepin, and steroids such as corticosteroids.

The above classes of drug products may undergo dosage titration or dosage adjustment, for which the subject invention can provide particular advantages, such as facilitating breakage of a whole or partial tablet into smaller doses to implement a treatment in accordance with a titrated or adjusted dose schedule

Another aspect of the invention is to utilize drug products that are manufactured in a dosing scheme that would lend itself to bisection or quadrisection, such as with four doses that are doubles of each other, such as 5, 10, 20, and 40 mg, tablets, but instead produce such products in a trisection fashion, either involving trisection of a known dosage strength such as a 40 mg tablet or a new dosage strength such as 22.5.

In a similar spirit to the above, certain drug products such as warfarin are produced for the U.S. market is dose strengths that are doubles, trebles, and quadruples of lower manufactured doses (e.g. 1, 2, and 4 mg tablets; and, 2.5, 5, and 7.5 mg tablets). The invention provides, as examples, for a trisected dosage strength that is not a treble dose of a lower manufactured dose, or for quadrisected dosage strength of a dose that is not a quadruple dose of a lower manufactured dose; or, the invention provides for quadrisection of known tablet strengths.

Also within the spirit and metes and bounds of the invention is a bisecting score of a drug product wherein the drug product has only one approved dose, such as Zetia® (10 mg), where it has not been taught or suggested to provide a bisected 10 mg or 20 mg tablet , or a drug product such as Proscar® (finasteride 5 mg), which has a different indication for the 1 mg finasteride product, marketed under the brand name Propecia.® In this instance, the subject invention can provide a quadrisected tablet which when broken can yield a 2.5 or 1.25 mg product, or a “pentasected” product which when broken into its indicated portions can yield five 1-mg products. Thus, yet another aspect of the invention relates to a score that guides division of a tablet into fifths, referred to herein as pentasected.”

In addition, the subject invention includes products which may be useful in a different dosage form (e.g., as tablets where the approved drug is marketed as a capsule) or strengths lower than currently or originally approved or marketed dosage strengths. For example, the subject invention can be advantageous where an orally dosed drug product is approved only in capsule form and its lowest available dose is higher than a minimal therapeutic amount. The advantages of the subject invention can thus be realized by providing that drug in a scored tablet, which is divisible into portions and, when broken or split in accordance with the divisions defined by the score pattern, is capable of providing lower strength doses from the whole tablet. Accordingly, the tablet portions can be used for treatments that call for the lower strength, or can be advantageous for adjusting (lose according to need of the patient or desire of the prescribing physician. Preferably, a drug product available only in capsule form or in certain minimum strengths in tablet form can be provided as a scored tablet produced in accordance with the configurations as described by Solomon and Kaplan, as referenced above.

These new dosage forms having novel score patterns can expand the utility of a drug product beyond what would be available from the available strengths or dosage forms. For example, doxepin HCl is marketed, in its lowest strength, as a 10 mg capsule. A drug product in accordance with the subject invention, such as a bisected or quadrisected doxepin HCl tablet can advantageously provide a variety of lower strengths that can be administered to a patient. For example, a 1 mg dose (trisected 3 mg tablet), 1.5 mg dose (bisected 3 mg tablet or trisected 4.5 mg tablet, 2 mg dose (bisected 4 mg dose tablet, trisected 6 mg dose tablet, or quadrisected 8 mg tablet), 2.5 mg dose (bisected 5 mg or quadrisected 10 mg tablet), 3 mg (quadrisected 12 mg tablet or bisected 6 mg tablet), and the like, can be made available and may further be advantageous for providing flexible dosing of a tabletted doxepin product. Specifically, doxepin or another like drug product, can thus be provided to a patient at a lower dose than contained in the whole tablet, and would not have been practicable from a previously available capsule dosage form because the capsule dosage form is not readily divisible into predictably accurate lower strengths. When the whole dosage form is provided as a dosage form that is divisible into predictably accurate lower doses, as in the tablet configurations described by Solomon and Kaplan, as referenced above, the subdivided lower doses in the tablet portions can be used for treatments that require such lower doses or strengths.

### BRIEF DESCRIPTION OF THE DRAWINGS

FIGS. 1(A)-1(C) depict embodiments of cross-scored compressed or molded tablets containing at least one drug, e.g., warfarin or doxepin, according to the subject invention: FIG. 1(A) shows a trisected tablet breakable into three portions; FIG. 1(B) shows a quadrisected tablet breakable into four portions; and FIG. 1(C) shows a pentasected tablet breakable into five portions.

FIGS. 2(A)-2(C) depict embodiments of compressed or molded tablets containing at least one drug, e.g., warfarin or doxepin, according to the subject invention and comprising a plurality of parallel scores provided on the same surface of the tablet: FIG. 2(A) shows a trisected tablet breakable into three portions; FIG. 2(B) shows a quadrisected tablet breakable into four portions; and FIG. 2(C) shows a pentasected tablet breakable into five portions.

FIGS. 3(A)-3(F) depict tablet scoring patterns, as in FIGS. 1(A)-1(C) and 2(A)-2(C), respectively, as formed in bi-layer compressed or molded tablets containing at least one drug, e.g., warfarin or doxepin, in accordance with embodiments of the subject invention.

### DETAILED DESCRIPTION OF THE INVENTION

Tablets of the invention are preferably those compressed in a conventional tablet press. For layered embodiments of the invention, a high-speed three (3)- or five (5)-station press produced by Korsch AG may be utilized. Remington's Pharmaceutical Sciences 20th Ed., Mack Publishing Co., Easton, Pa. (2000), which is incorporated by reference in its entirety, describes in Chapter 45 the various techniques utilized in making compressed tablets. Conventional formulation procedures, including the use of mixtures, powders, granulations, pellets, and like, as well as known, pharmaceutically acceptable excipients can also be employed in the manufacture of tablets of the subject invention. Tablets of the invention are primarily intended for oral administration but they may also be used for other applications. As is also known in the pharmaceutical arts, formulations and tablets may be coated or may be contained within a capsule.

The subject invention can be readily understood by describing specific examples, which are intended as illustrative of the invention, and are not intended as limiting. As one example, warfarin sodium may be usefully produced as a trisected, quadrisected, or pentasected tablet, which can be accurately broken into predictable partial doses. If the tablet were taken whole, such a scoring pattern would be irrelevant. Preferably, the quadrisected warfarin tablet can be provided as a tablet which is manufactured according to the techniques, and to provide the advantages of the tablets disclosed in WO 2005/112900 and WO 2006/038916. A trisected product may also be produced according to known techniques with a five layer tablet such as one that can be produced with the Korsch TRP 900, in which, for example, the first, third, and fifth layers (segments) comprise equal and therapeutic amounts of warfarin sodium, and the second and fourth layers (segments) lack significant amounts of warfarin sodium.

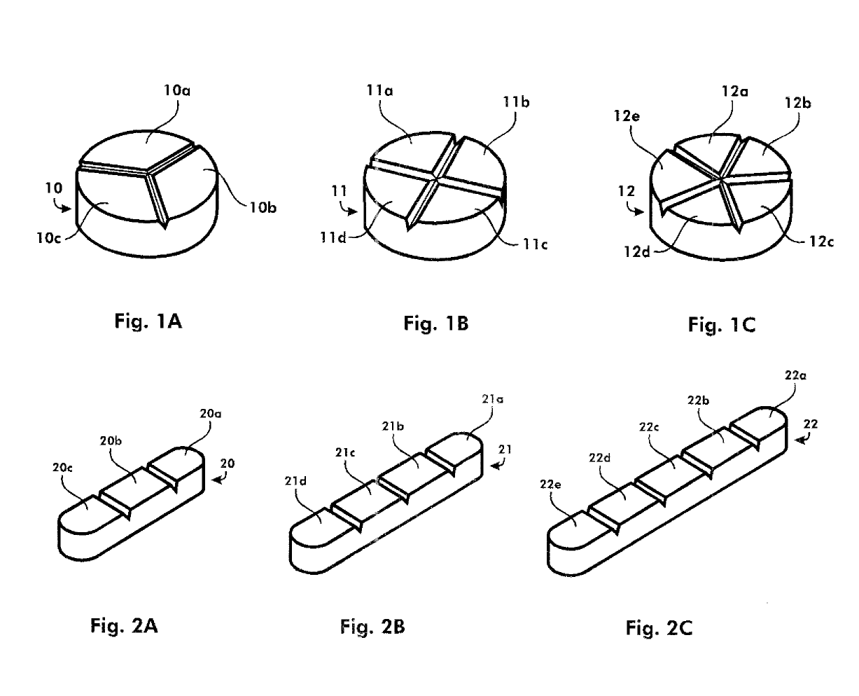

Another example involves the antihypertensive agent torsemide, which has favorable properties at low doses. A trisected 6 mg tablet would be novel and useful, especially if accurately divisible into three 2 mg tablettes. Also, a useful and advantageous composition considered part of the subject invention is a quadrisected 8 mg torsemide tablet, breakable into four 2 mg “tablettes.”. A quadrisected 4 mg torsemide tablet would be useful to produce the known effective antihypertensive doses of 2, 3, and 4 mg doses; and the 1 mg tablet can be useful as a low dose alternative to provide antihypertensive efficacy in cases of an adverse reaction to a higher dose or in pediatric cases, and may have activity against hypertension in some patients. Torsemide tablets according to the subject invention may be provided as scored tablets embodied as shown in FIGS. 1, 2, or 3, wherein the tablet comprises torsemide as the active ingredient rather than warfarin.

In another example, the anticonvulsant lamotrigine is currently produced as a bisected tablet including a 100 mg dose, but is instructed to be taken whole. It would therefore be advantageous to produce a quadrisected 100 mg lamotrigine tablet if predictable 25 mg dosages (tablettes) can readily be obtained by quartering said tablet according to the quadrisect pattern. Lamotrigine tablets according to the subject invention may be provided as scored tablets embodied as shown in FIGS. 1, 2, or 3, wherein the tablet comprises lamotrigine as the active ingredient rather than warfarin.

Another example involves the pharmaceutical agent doxepin, which has favorable properties at low doses. A trisected 3 mg tablet would be novel and useful, and preferably if accurately divisible into three 1 mg tablettes. Also, a useful and advantageous composition considered part of the subject invention is a quadrisected 8 mg tablet, breakable into four 2 mg “tablettes.” A quadrisected 4 mg doxepin tablet would be useful to produce the known effective doses of 1, 2, 3, and 4 mg doses; and the 1 mg tablet can be useful as a low dose alternative to provide efficacy in cases of an adverse reaction to a higher dose or in pediatric cases, and may additional activity in some patients. Doxepin tablets according to the subject invention may be provided as scored tablets embodied as shown in FIGS. 1, 2, or 3, wherein the tablet comprises doxepin as the active ingredient and is manufactured using known tabletting techniques and employing conventional, pharmaceutically acceptable excipients.

Turning to the Figures, FIGS. 1(A)-1(C) depict embodiments of cross-scored substantially round or ovoid tablets containing an active pharmaceutical ingredient (API) according to the subject invention. FIG. 1(A) shows a trisected tablet 10 containing an API, e.g., warfarin or doxepin, composition, wherein the tablet is breakable along the score lines into one or more of the three portions 10a, 10b and 10c. FIG. 1(B) shows a quadrisected tablet 11 breakable along the score lines into one or more of the four portions 11a, 11b, 11c, and 11d. FIG. 1(C) shows a pentasected tablet 12 breakable along the score lines into one or more of the five portions 12a, 12b, 12c, 12d, and 12e.

FIGS. 2(A)-2(C) depict embodiments of substantially elongated, or capsule shaped tablets according to the subject invention, comprising a plurality of parallel scores provided on the same surface of the tablet. FIG. 2(A) shows a trisected tablet 20 breakable along the score lines into one or more of the three portions 20a, 20b, or 20c. FIG. 2(B) shows a quadrisected tablet 21 breakable along the score lines into one or more of the four portions 21a, 21, b, 21c, or 21d. FIG. 2(C) shows a pentasected tablet 22 breakable along the score lines into one or more of the five portions 22a, 22b, 22c, 22d, or 22e.

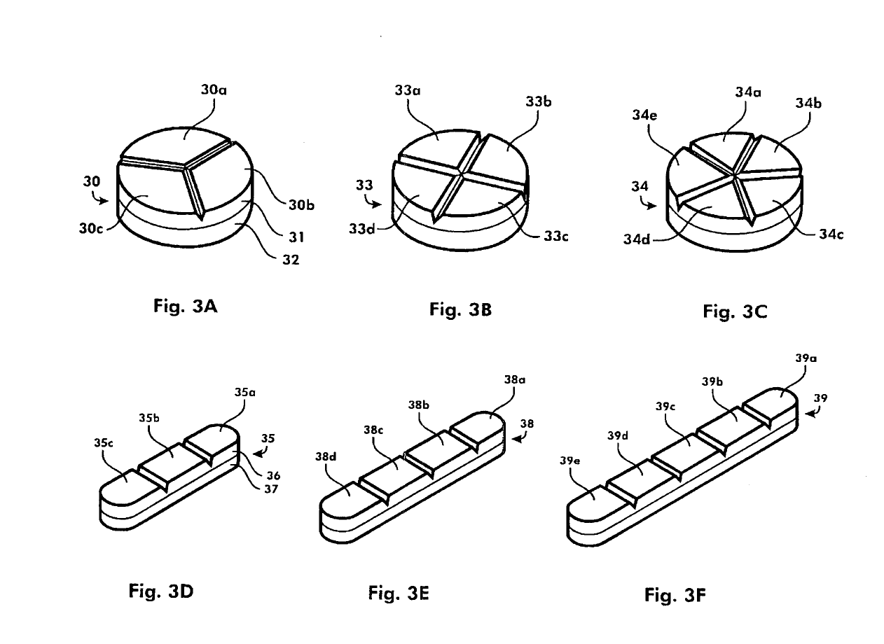

FIGS. 3(A)-3(C) depict tablet scoring patterns formed in substantially round or ovoid bi-layer tablets containing an API such as doxepin or warfarin, in accordance with the subject invention. FIGS. 3(D)-3(F) depict tablet scoring patterns formed in substantially elongate or capsule shaped bi-layer tablets in accordance with the subject invention. FIG. 3(A) shows a trisected bi-layer tablet 30 containing API in a first layer 31 and an inactive composition in a second layer 32. The bi-layer tablet 30 is breakable along the score lines into one or more of the three bi-layer portions 30a, 30b or 30c. FIG. 3(B) shows a quadrisected bi-layer tablet 33 containing an API and breakable along the score lines into one or more of the four portions 33a, 33b, 33c, and 33d. FIG. 3(C) shows a pentasected bi-layer tablet 34 breakable into one or more of the five portions 34a, 34b, 34c, 34d, or 34e. FIG. 3(D) shows a trisected bi-layer tablet 35 containing API in a first layer 36 and an inactive composition in a second layer 37, the bi-layer tablet being breakable along the score lines into one or more of the three portions 35a, 35b, or 35c. FIG. 3(E) shows a quadrisected bi-layer tablet 38 containing API in the scored layer and breakable along the score lines into one or more of the four portions 38a, 38b, 38c, or 38d. FIG. 3(F) shows a pentasected bi-layer tablet 39 containing API in the scored layer and breakable along the score lines into one or more of the five portions 39a, 39b, 39c, 39d, or 39e.

Other drug products, e.g., drug products comprising any of the active ingredients described herein, can be embodied in tablets uniquely scored with the subject score patterns to advantageously provide predictably accurate lower doses to a patient. Accordingly, the examples and the accompanying drawings are not intended to be limiting, and are provided for illustration purposes only. It is recognized that related inventions may be within the spirit of the disclosures herein, and no omission in this application is intended to limit the inventors to the current claims or disclosures. While certain preferred and alternative embodiments of the invention have been set forth for purposes of disclosing the invention, modifications to the disclosed embodiments may occur to those who are skilled in the art.

1. An improved pharmaceutical tablet containing a drug wherein said drug is provided in the prior art as a capsule, an unscored tablet, or a tablet having a bisected scoring pattern, said improvement comprising:
a score pattern in or on said tablet selected from the group consisting of a trisecting, quadrisecting, aid a pentasecting score pattern.

2. A trisected or quadrisected pharmaceutical tablet chosen from the groups of pharmaceutical Products listed in Section 3 of the 2006 Physicians Desk Reference, except for the quadrisected and bisected/trisected tablets listed in the Background of the Invention.

3. The trisected or quadrisected tablet of claim 2, said tablet comprising a psychotherapeutic agent.

4. The tablet of claim 3, wherein said psychotherapeutic agent is doxepin or a salt, isomer, polymorph, hydrate, prodrug, or derivative thereof.

5. The tablet of claim 4 wherein said active pharmaceutical ingredient is doxepin HCl.

6. A method of treatment for a patient in need of a psychotherapeutic agent, said method comprising:
providing a tablet comprising a psychotherapeutic agent, said tablet having a scoring pattern therein, said scoring pattern selected from the group consisting of a bisecting, trisecting, quadrisecting, and a pentasecting score pattern.
providing instruction to the patient to break the tablet in accordance with the scoring pattern as needed for providing a dose different than contained in a whole tablet: and
administering to the patient a portion of said tablet.

7. The method of claim 6 wherein said administration of the tablet portion provides a lower dose than provided in a whole tablet.

8. The method of claim 6 wherein said administration of the tablet portion provides a dose that is an intermediate dose between a dose provided in a whole tablet.

9. The method of claim 6 wherein said psychotherapeutic agent is doxepin or a salt, isomer, polymorph, hydrate, prodrug, or derivative thereof.

10. The method of claim 9 wherein said psychotherapeutic agent is doxepin HCl,

11. An article of manufacture comprising: (a) a tablet or tablets comprising a psychotherapeutic agent, said tablet or tablets having a scoring pattern therein, said scoring pattern selected from the group consisting of a bisecting, trisecting, quadrisecting, and pentasecting score pattern, and (b) a separate instruction instructing that the use of the tablet or tablets may be broken in accordance with the scoring pattern to provide a dose which is different than provided in a whole tablet or tablets wherein said tablet or tablets and instruction are packaged together.

12. The article of manufacture of claim 11 wherein said different dose provided by breaking of said tablet or tablets is a dose lower than contained in a whole tablet.

13. The article of manufacture of claim 11 wherein said different dose provided by breaking of said tablet or tablets is an intermediate dose not contained in a whole tablet or tablets.

14. The article of manufacture of claim 11 wherein said psychotherapeutic agent is doxepin, or a salt, isomer, polymorph, hydrate, prodrug, or derivative thereof.

15. The article of manufacture of claim 14 wherein said psychotherapeutic agent is doxepin HCl.
